(19) United States
(12) Patent Application Publication
(10) Pub. No.: US 2010/0292545 A1
(43) Pub. Date: Nov. 18, 2010

(54) INTERACTIVE PSYCHOPHYSIOLOGICAL PROFILER METHOD AND SYSTEM

(75) Inventors: Chris Berka, Carlsbad, CA (US); Daniel J. Levendowski, Carlsbad, CA (US); Philip R. Westbrook, Fallbrook, CA (US); Gene Davis, Oceanside, CA (US); Robin Johnson, Oceanside, CA (US); Djordje Popovic, Oceanside, CA (US)

Correspondence Address: PROCOPIO, CORY, HARGREAVES & SAVITCH LLP, 525 B STREET, SUITE 2200, SAN DIEGO, CA, 92101, US

(73) Assignee: ADVANCED BRAIN MONITORING, INC., Carlsbad, CA (US)

(21) Appl. No.: 12/466,263

(22) Filed: May 14, 2009

(51) Int. Cl. A61B 5/0205

(52) U.S. Cl. 600/301

(57) ABSTRACT

An efficient, objective, flexible and easily deployable system for conducting evaluations of mental and physiological state and recommending individualized treatment to improve said state is described. The method and system are based on commensurate measurement of mental functions, levels of stress and anxiety, and/or biologically active molecules such as neurotransmitters, immune markers including cytokines and hormones. The method and system are designed to assess an individual's cognitive function and the underlying physiology in order to delineate various disease processes, injuries, drug states, training stages, fatigue levels, stress levels, aging processes, predict susceptibility to stress and/or sleep deprivation, identify aptitude for training and/or characterize effects of any experimental conditions. The system and method may be used in recommending individualized treatment protocols, as well as to guide the treatment process by assessing the efficacy of such therapies in the clinical trials process.

### STATEMENT REGARDING FEDERALLY SPONSORED RESEARCH OR DEVELOPMENT

This invention was made with U.S. government support under one or more of the following contracts: NIH NIMH grant number MH078436 and NIH NHLBI grant number HL70484 awarded by the National Institute of Health. The U.S. government has certain rights in the invention.

### BACKGROUND OF THE INVENTION

1. Field of the Invention

The present invention relates generally to neuropsychological and psychophysiological tests for measuring and characterizing mental and/or emotional function, or changes in function; and more particularly to the use of electroencephalogram (“EEG”) and other measures to profile an individual's psychophysiological condition.

2. Description of the Related Art

Alterations in neurocognitive function often play a major role in numerous sleep, neurological and psychiatric disorders and can have a significant impact on quality of life, treatment efficacy, rehabilitation and ability to function at work and in normal daily activities. Assessment of an individual's cognitive capacity is important for diagnosing and objectively evaluating putative treatments for any condition that affects higher brain functions (e.g., identifying cognitive deficits associated with sleep apnea and evaluating changes in cognitive after treatment with drugs or devices). A barrier to comprehensive assessment is, however, the lack of efficient, affordable and comprehensive tests which adequately profile an individual's entire psychophysiological condition.

Conventional neuropsychological assessment (e.g., Halstead-Reitan, WAIS, Wechsler memory battery) is time-consuming, expensive and require substantial technical expertise to administer and interpret. Computerized behavioral tests such as the Automated thousands of clinical trials or the commercially available CANTAB, Continuous Performance Test (“CPT”) or Psychomotor Vigilance Test (“PVT”) can reduce costs and shorten the time needed for taking a test, but they still fail to assess neurocognitive functions directly and to control or account for motivational, emotional and other relevant factors. Both conventional and computerized neuropsychological tests indirectly assess cognitive function by measuring the subject's performance. Interpretation of the results and/or comparison of performance results within and among subjects can be insensitive or unspecific because motivation, emotional state (e.g., level of anxiety), fatigue, concomitant physical illness or use of psychoactive substances are difficult to control and all impact performance. Changes in other physiological systems and/or mechanisms, such as endocrine or immune, can thoroughly and often unpredictably influence cognitive functions, masking or distorting the effect of recovery or decline of a function.

Cognitive function can be assessed in a direct manner by measuring the brains electrical activity (e.g., EEG, fMRI, fNIR, MEG, etc.). Most references which use EEG rely on simple and conventional measures, such as EEG alpha, theta or delta band power; P300 evoked potential amplitude or EEG coherence measures. Some examples include: 1) U.S. Pat. No. 4,203,452 to Cohen, where a single channel EEH is measured in an attempt to ascertain if a student is undergoing short- or long-term learning; 2) U.S. Pat. No. 5,339,826 to Schmidt et al., where the effectiveness of video-taped training material is tested analyzing a student's alpha and beta EEG activity as well as event-related potentials during multiple choice questions; 3) U.S. Pat. No. 3,809,069 to Bennett, where the intelligence of a subject is measured using pulsed stimuli to evoke subject responses which are then compared to the frequencies of responses of others; and 4) U.S. Pat. No. 5,447,166 to Gevins, where EEG signals are used to alter a computer program and present more or less difficult test material to the user depending on their level of alertness and distractibility. These direct measures of cognitive function share the same limitations as the indirect measures, in that for a given task, motivation, emotional state, fatigue, illness or pharmaceutical substances contribute to substantial within- and among-subject differences that compromise the accuracy of the quantified results and makes the interpretation of the results at best subjective. Without adequate controls, tracking changes in cognitive functions due to disease or treatment, for example, is limited. In addition to the above mentioned limitations, fMRI and MEG are impractical for routine assessment because they require cumbersome equipment and technical expertise to operate. Reliance on acquisition of baseline data from a fully-rested individual to determine the subject's alertness, for example, can be unreliable, because daytime drowsiness is a symptom of a number of disease states (e.g., obstructive sleep apnea).

Although there is scientific literature that correlates indirect and direct measures of neurocognitive function in various populations under multiple conditions, Gevins et al. (U.S. Pat. No. 6,947,790) first proposed to combine these two into a single metric and to measure changes in the NeuroCognitive Factors Change (“NCFC”) score in a test-retest paradigm. An aspect taught by Gevins was the need to acquire and store the direct measures of cognitive function (e.g., EEG, fMRI, MEG) in a manner that provides precise synchronization with the indirect measures (e.g., behavioral performance). Certain analyses such as the P300 are reliant on the point in which the stimulus or response occurs is accurately marked in the EEG file for accurate detection of the electrophysiological patterns. Gevins shows that a NCFC score could be obtained by applying a multivariate analysis to a variety of conventional measures of EEG (alpha, theta, delta power and N100 and P300 evoked potentials) while the subject was performing different tests designed to evaluate memory, attention, and overall EEG patterns during non-specific tasks (e.g., eyes closed). Gevins requires that the subject be tested at least twice, once at baseline and again after a change in condition (e.g., post-treatment or post-pharmaceutical intervention). Finally, Gevins suggests that the analytical technique used to compute the NCFC score first required an expert to establish thresholds that could be based on information derived from a normative database.

The present application addresses shortcomings in the references by providing unique means to characterize degradations in sustained attention, alertness, and verbal and spatial memory and combines these degradations with other non-neurocognitive measures that can quantify measures from a single study/test or characterize changes in each of the multitude of measures when used in a test-retest paradigm.

### SUMMARY OF THE INVENTION

A method and system which incorporates a neurocognitive test bed that acquires a multitude of physiological signals synchronized with behavior/performance data (“neurocognitive data”) under a multitude of test conditions and analyzes neurocognitive data and derives multitudes of neurocognitive measures from the same data is disclosed. In some aspects, the method and system further combine the neurocognitive measures with anthropometric measures and physiological measures to derive a comprehensive profile of the subject psychophysiological condition.

In one embodiment, a method of determining a subject's cognitive and emotional state includes obtaining a first physiological measure from the subject which provides baseline data, administering to the subject a test including a plurality of self-paced computerized tasks while obtaining a second physiological measure from the subject which provides task-related data, deriving one or more scores quantifying the subject's cognitive and emotional states utilizing the task-related data, and creating a profile by determining the significance of the one or more scores with reference to normative data from a plurality of subjects.

In another embodiment, a method of creating a physiological profile for a subject includes obtaining a first physiological measure from the subject which provides baseline data, administering to the subject a test including a plurality of self-paced computerized tasks while obtaining a second physiological measure from the subject which provides task-related data, deriving a plurality of scores quantifying the subject's physiological state utilizing the task-related data, determining a diagnosis based on the plurality of scores and normative data from a plurality of subjects, and generating the physiological profile for the subject, the physiological profile including the diagnosis.

In another embodiment, a system for use in determining a subject's cognitive and emotional state includes a sensor in communication with the subject, a display in communication with the subject, and a processor in communication with the sensor and display, the processor configured to: obtain a first physiological measure from the sensor which provides baseline data; provide to the display a test including a plurality of self-paced computerized tasks while obtaining a second physiological measure from the sensor which provides task-related data; derive one or more scores quantifying the subject's cognitive and emotional states utilizing the task-related data; and create a profile by determining the significance of the one or more scores with reference to normative data from a plurality of subjects.

### BRIEF DESCRIPTION OF THE FIGURES

The details of the present invention, both as to its structure and operation, may be gleaned in part by study of the accompanying drawings, in which like reference numerals refer to like parts, and in which:

FIGS. 1A and 1B are a schematic representation of a system for performing interactive psychophysiological profiles, according to an embodiment.

FIGS. 2A-B are graphical representations of: (A) mean percent correct answers and (B) B-Alert percentage classified drowsy for 20-min 3C-VT at 5-min intervals for Experiment 1.

FIGS. 3A-D are graphical representations of: (A) mean measures for three groups stratified based on performance across 44-hours of sleep deprivation, (B) reports of subjective sleepiness, (C) % of correct responses, and (D) time course of B-Alert EEG drowsiness classifications for Experiment 1.

FIGS. 4A-D are graphical representations of the measurement of the P300 amplitude for Experiment 2.

FIGS. 5A-B are graphical representations of changes in event-related potentials caused by sleep deprivation over a 4-week period: (A) stratified by vulnerability group and (B) average difference by vulnerability group for Experiment 2.

FIG. 6 is a graphical representation of distribution of ACES for OSA and healthy subjects for Experiment 3.

FIG. 7 is a graphical representation of a comparison of OSA patients pre-treatment vs. 1 month post-treatment for Experiment 3.

FIGS. 8A-D are graphical representations of calculated mean neurocognitive factor scores for: (A) processing speed, (B) visual memory, (C) sustained attention, and (D) verbal memory for Experiment 3.

### DETAILED DESCRIPTION OF THE INVENTION

After reading this description, it will become apparent to one skilled in the art how to implement the invention in various alternative embodiments and alternative applications. The following description sets forth numerous specific details, such as examples of specific systems, components and methods in order to provide a good understanding of several embodiments of the present invention. It will be apparent to one skilled in the art, however, that at least some embodiments of the present invention may be practiced without these specific details. In other instances, well-known components or methods are not described in detail or are presented in simple block diagram format in order to avoid unnecessarily obscuring the present invention. Particular implementations may vary from these exemplary details and still be contemplated to be within the spirit and scope of the present invention.

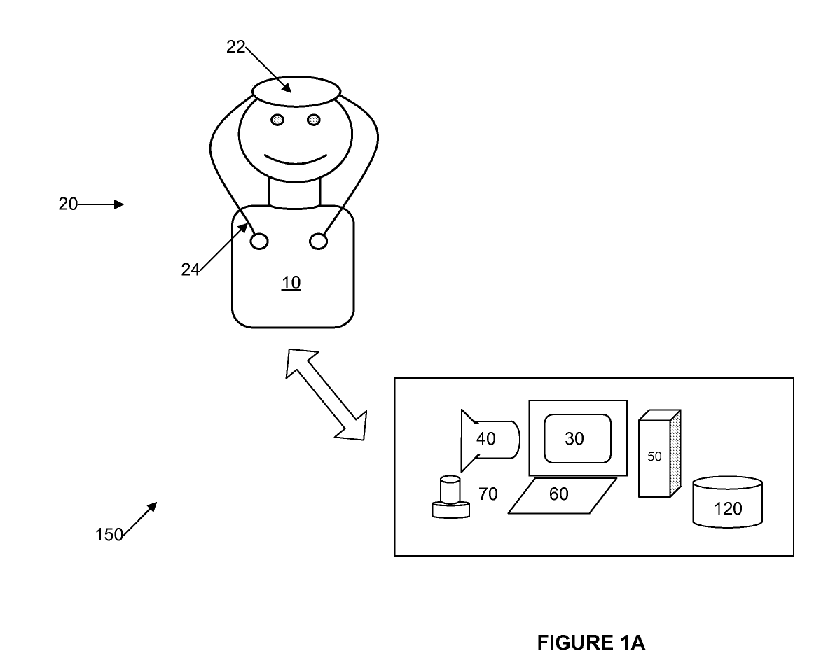

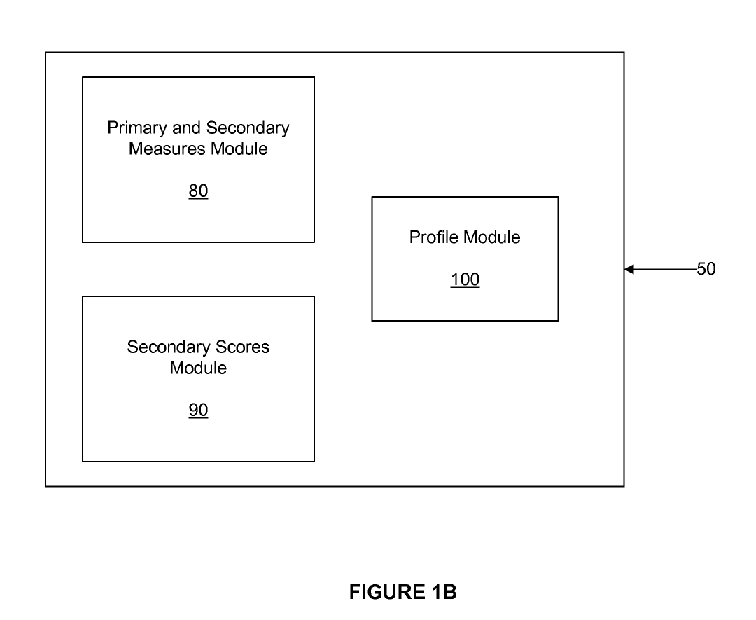

Turning now to FIG. 1, a system 150 for use with a human subject 10 is shown. In an embodiment, a device 20 worn by subject 10 includes a headset 22 having EEG electrodes (not shown) which contact the scalp of the subject, with optional leads for EKG electrodes 24 and other sensors for monitoring physiological signals which contact the appropriate locations on the subject's body. A suitable headset 22 is described in U.S. Pat. Nos. 6,161,030 and 6,381,481, herein incorporated by reference. In one embodiment, EEG is acquired from three, six or nine sites from a patient's head to reduce the time required to prepare the individual for a study, however, any number of electrodes may be used.

According to one embodiment, headset 22 communicates with, and transmits acquired physiological signals to, a computer 50 in a wireless manner to reduce signal contamination. Conventional systems for physiological signal acquisition, including in-laboratory EEG and/or EKG systems, and wired transmission of the acquired data from sensors to the computer may alternatively be employed.

In some embodiments, physiological signals affected by or reflective of cognitive functions or emotional states can be acquired along with the EEG and/or EKG. These physiological signals may include electrical activity of skeletal muscles (e.g., electromyography—“EMG”), eye movements (recorded with standard electrooculography, “EOG”, or with an optical eye-tracking system based on a camera), respiration (recorded, for example, with an oro-nasal thermistor, or a nasal cannula connected to a pressure transducer, or with a respiratory piezo-band, etc.), brain oxygenation (measured with a functional near-infrared spectroscopy, “NIRS”), tissue oxygenation (measured with a pulse oximeter), electrical conductivity of the skin (galvanic skin reaction, “GSR”), blood flow through regions of interest such as the brain (measured with Doppler ultrasound, or with fMRI) and any other physiological signal that changes as a function of neurocognitive or emotional state. For example, it is recognized that the intensity, quality and frequency of eye movements change as a function of the level of alertness/drowsiness of a subject. Intense and rapid scanning eye movements, typical for active wakefulness, become less frequent and the ocular activity in general gradually decreases as the subject becomes fatigued/drowsy, until a different type of regular, sinusoidal eye movements, the so called Slow Eye Movements (“SEM”), occur indicating the onset of the initial stage of sleep. The impeding drowsiness leads to a notable decrease in the volume of respiration as compared with the preceding state of active wakefulness, and sometimes to slow sinusoidal variations (with a period of 90-120 seconds) in peak-to-peak amplitudes of breaths. Irregular respiration can on the other hand be a sign of arousal, anxiety, or other strong emotions. Galvanic skin reaction (“GSR”), EMG of selected muscle groups (e.g., neck, facial musculature, forearm and/or hand), size of the pupil and facial expression (both captured with a small camera), and heart rate measured with a pulse oximeter are commonly used to make inferences about emotional state of users of computers/computer systems. Task-related variations in oxygenation and/or blood flow through different parts of the brain are commonly used (such as in fMRI studies) to identify brain regions involved in or crucial for the execution of the studied task. Those skilled in the art will recognize that any number physiological signals that change as a function of a cognitive or emotional state, and any number of corresponding sensors can be used for this purpose.

In one embodiment, a biological sample can also be obtained from subject 10, and physiological data (e.g., the concentration and/or activity) about molecules such as certain hormones and cytokines, whose concentration or activation is known to change as a function of cognitive or emotional states, or of disorders or diseases affecting cognitive and emotional states can be determined. For example, it is known that long-lasting sleep restriction increases blood levels of cortisol; stress and anxiety increase blood levels of both cortisol and adrenaline; depression affects blood levels of thyroid hormones, cortisol and estrogen, and is associated with decreased tissue levels of the neurotransmitter serotonin, etc. Additionally, immune markers (such as IL-1, CRP, macrophage migration inhibitory factor, or immunoglobulin) have been shown to be elevated in cerebrospinal fluid (“CSF”), blood, or in samples of brain tissue of subjects with autistic spectrum disorder (“ASD”), schizophrenia or attention deficit, hyperactivity disorder (“ADHD”), and depression. Those skilled in the art will understand that the relationship between cognitive and emotional states or disorders on one hand, and endocrine and immune systems on the other hand, is far more complex than the afore presented examples can illustrate, and therefore the aforesaid examples should by no means limit the scope of this invention. These and other biologically active molecules of interest can be detected in blood, urine, saliva, sweat, or hair using any standard method (ELISA, HPLC, Multiplexing or Flow Cytometry). While obtaining blood, urine, saliva or sweat samples is relatively simple and either non-invasive or minimally invasive, the current state of technology does not allow for an easy collection of CSF or routine brain biopsies. However, with future improvements in the related technology ambulatory collection of CSF or brain tissue samples may become reality. Once the physiological data are derived, the results can be combined with the physiologic and/or anthropometric data for further analysis.

### Data Acquisition:

In one embodiment, physiological signals (e.g., subject's brain waves, heart electrical activity, etc.) are simultaneously acquired while the subject is presented with tasks that test cognitive functions, such as learning speed and aptitude, verbal and visuospatial memory, sustained attention, selective attention, distractibility, impulsiveness, adaptability, flexibility in responding, speed in responding and propensity for risk-taking, etc. A series of trials of easy and more difficult versions of one or more of the tasks is presented. For comparison, the subject's 10 brain waves (and/or other physiological signals) are also recorded briefly while he or she rests both with eyes open and eyes closed. The task is presented on the screen 30, and/or by a loudspeaker 40 that are both connected to a computer 50 such as a personal computer or mobile device. Subject 10 responds using keyboard keys 60 or other input/output device 70 such as a switch, joystick or mouse. Portable hardware and/or software technologies preferably provide self-paced instructions to the user before each task, monitor responses during train-to-criterion sessions and conduct routine impedance monitoring to ensure high quality physiological data acquisition. For example, in some embodiments, such as when performing field tests, the physical data acquisition is performed on portable equipment. In an embodiment, the resultant test battery acquires the maximum amount of neurobehavioral and neurophysiological data as efficiently as possible. Examples of these tests used include:

1) The 3-Choice Vigilance Task (3C-VT) incorporates features of the most common measures of sustained attention, such as the Continuous Performance Test, Wilkinson Reaction Time, and the PVT-192, and it was designed to allow simultaneous monitoring and quantification of the EEG. The 3C-VT, generally administered as a first Alertness and Memory Profiler (“AMP”) test, requires subjects to discriminate one primary (70%) from two secondary (30%) geometric shapes presented for 0.2 seconds over a 20-minute test period. A training period is provided prior to the start to minimize practice effects. Concurrent validity was established in sleep deprivation studies by correlation with behavioral evidence as measured by: cessation of finger tapping; visually scored observations of facial signs of drowsiness (eye closures, head nods); responses to a subjective sleepiness questionnaire; visually scored EEG; modified MWT; handheld PVT-192 test; and driving simulator performance.

2) A 5-minute Eyes Open (“EO”) and Eyes Closed (“EC”) with paced button press. EO presents a 10 cm circular image every two seconds for 200 milliseconds in the center of the monitor. Participants are instructed to press the space bar each time they see the image. For EC, an auditory tone every 2 seconds prompts participant to press the space bar.

3) An Image Recognition Learning and Memory Tests (“IR”) evaluate attention, distractibility, and encoding and image recognition memory. For the Standard IR, during the training session, a group of 20 images are presented twice. The testing session presents the 20 training images randomly interspersed with 80 additional images. Subjects must indicate whether or not the image was in the training set. Five equivalent image categories are available including animals, food, household goods, sports, and travel. For the Interference IR, a set of 20 new images must be memorized and distinguished from the first set of training images and 60 images previously displayed in the Standard PAL. In Numbers IR, a number is assigned to each image and subjects must identify the correct image-number pairs.

4) The Verbal Paired Associate test (“VPAL”) is identical to the image recognition, substituting word pairs for images. During training, 20 word pairs are presented twice. During testing, the 20 training pairs are randomly interspersed with 80 new word pairs. Subjects indicate whether or not the word pair was in the training set. Easy (e.g., black-white) and difficult (e.g., table-horse) word pairs are included in each test.

5) The Verbal Memory Scan (“VMS”) is a Stemberg serial probe recognition memory task developed to measure speed and accuracy of verbal working memory. Lists of 3, 5, or 7 words (memory sets) are presented, followed by single-word probes (in-set or out-of-set). Stimulant drugs including caffeine, nicotine, and amphetamine have been shown to improve performance in this test, increasing the efficiency of memory-search as measured by both behavioral and EEG data.

6) The Forward and Backward Digit Span are similar to those in the Wechsler Memory Test. In Forward Digit Span, subjects are presented with a series of single digits of varying lengths and asked to type the series in the order that it was presented. For Backward Digit Span, subjects are presented with a series of single digits of varying lengths and asked to type the digits in the reverse order from the one in which they were presented. These tasks require subjects to employ verbal working memory resources.

Those skilled in the art will recognize that any choice of test set is to some extent arbitrary, thus any number of available standard psychometric tests can be used as part of the test battery or that new tests could be created to elicit the distinct cognitive attributes or responses which would be beneficial in classifying the neurocognitive state.

In one embodiment, the tests are administered to subject 10 on monitor or display screen 30 of computer 50. In another embodiment the tests are administered to subject 10 on a screen of a mobile communication device such as PDA or iPod, or any other similar device provided that the screen has a sufficient size and resolution.

In an embodiment, the tests are stored in the memory of the same computer 50 or mobile device that presents the tests to the subject 10, communicates with the data acquisition device 20, and executes the tests or algorithms used in modules 80 and 90. Yet, in another embodiment, the tests as well as algorithms used in modules 80 and 90 can be stored and/or executed on a server that can be accessed via Internet (not shown). In one embodiment, module 80 represents a module for determining performance measures. For example, module 80 may include primary and secondary measures derived from physiological signals acquired from subject 10.

In another embodiment, module 90 represents a module for determining a variety of secondary scores that quantify the subject's cognitive functions and/or emotional states. For example, module 90 may include a mathematical model or algorithm that manipulates or performs operations on historical data such as demographic, anthromorphic, and clinical data acquired from subject 10.

In some embodiments, the subject 10 that uses the computer 50 or mobile device to connect to the server computer that administers the tests to the subject 10 uses computer 50 as a slave device. In this embodiment, computer 50 communicates with the data acquisition system 20, and sends the acquired data to the server computer so that the data can be processed in the way described further below.

It should be understood that some or all of these tests can have several versions of gradually increasing degree of difficulty. For example, the Verbal Memory Scan (VMS) test can be performed with 3, 5 or 7 words in the list to be memorized, or the Verbal Paired Associate (“VPAL”) test can contain different proportions of the easy (e.g., ‘black-white’) and difficult (e.g., ‘horse-table’) word pairs. Different versions of the same test can be used to assess the subject's 10 learning abilities and adaptability. For example, the subject 10 can first be administered an easy version of a test (e.g., the VMS with 3 words), which is followed by presenting the subject with increasingly difficult versions of the same test (e.g., the number of words to memorize increases gradually from 3 to 8). The present system can track changes in performance from the easiest to the most difficult tests (% of correct responses, time taken to solve each problem) as well as changes in the concomitantly acquired physiological signals (EEG, EKG, and so forth), and use these changes to derive one or few quantitative descriptors of the learning ability or adaptability in a way described further below.

In addition to the aforesaid tests and other tests known from experimental and clinical psychology, the subjects 10 can also be presented with tasks that require no cognitive effort or emotive engagement. One example of such tasks includes passively sitting with eyes closed and eyes open as it is routinely done during calibration prior to EEG recording. These pseudo-tests can serve as baseline conditions (“flat line”) for the acquisition of the physiological signals, to which all other recordings made during tests that do engage the subject's 10 cognition or emotions are normalized. The “normalization” can be performed as mere comparison of the test recordings to the baseline condition recordings and computing a difference or a ratio of the two measurements; or it can involve a more mathematically involved normalization procedure such as Z-transform.

### Data Analysis:

In some embodiments, a plurality of primary measures is computed in module 80 from the performance data including:

1) Mean, standard deviation and variability of the subject's reaction time to each task trial;

2) Mean, standard deviation and variability of the accuracy of the subject's response to each task trial (the measures 1 and 2 will theretofore be referred to as behavioral or performance measures);

3) Power spectral density (“PSD”) of the subject's EEG activity recorded from multiple scalp sites, computed on a second-by-second basis for the range of frequencies from 1 to 50 Hz in 1 Hz steps. Power can be computed using standard non-parametric algorithms (Fast Fourier Transform, filter banks, and so forth) or parametric techniques such as auto-regressive (“AR”) models or autoregressive moving-average (“ARMA”) models. The computed PSD indices may further be grouped or summed into the conventional EEG bands known from the literature, namely delta (1-4 Hz), theta (4-7 Hz), alpha (8-13 Hz), beta (13-30 Hz) and gamma (30-50 Hz). Specific changes in PSD of the EEG across different scalp sites can serve as measures of alertness, mental effort and engagement, workload imposed on the subject by a task, and the subject's working memory as it is described in detail in the remainder of this document (see below the Linear Drowsiness Scale, Workload metrics, and Experiments 1 and 2).

4) Peak times (latencies), peak amplitudes and wave shapes of the subject's event-related potentials (“ERPs”) elicited by the task stimuli (including the N100, as N100, P200, P300, N400, the Memory-Related Late Positive component, and other event-related potentials described in the relevant literature). The aforesaid potentials can be timed to the times of occurrence of known stimuli in the tests/tasks presented to the subject (Stimulus-related ERPs), or to the times of occurrence of the subject's behavioral responses to the presented task/test, such as a button press (Response-related ERPs), or to a physiological event such as eye gaze fixation (Fixation-related ERPs, or “FLERPs”) that can be tracked by an optical eye tracker with a camera, or by means of EOG. Experimental literature has demonstrated that latencies, amplitudes and overall shape of event-related potentials is affected by, and can therefore reflect, perceptual acuity and speed, decision-making speed and accuracy, capacity for selective, focused and global attention, or situational awareness. The PSD computations described in paragraph 3) above can be combined with ERPs (whether stimulus- or response-related, or FLERPs) into power event-related potentials (“PERPs”), which are the PSD computed as described above on a segment of EEG that contains ERP(s) centered to either a stimulus, or response or a physiological event such as eye fixation, or a heartbeat.

5) Instantaneous heart rate, computed from the time interval between two successive QRS complexes in the recorded EKG, and the heart rate variability (“HRV”) signal, computed as the first difference of the instantaneous heart rate;

6) Power of the HRV signal in two ranges, the low frequency (“LF”) range from 0.04 to 0.15 Hz and high frequency (“HF”) range from 0.15 to 0.4 Hz. Power can be computed using standard non-parametric algorithms (Fast Fourier Transform, filter banks, and so forth) or parametric techniques such as auto-regressive (“AR”) models or autoregressive moving-average (“ARMA”) models. Once the LF and HF power is computed, they can be presented as percentages of the total power, or a ratio of the LF to HF power can be computed. The LF power and the LF/HF ratio have been shown to be related to the degree of sympathetic stimulation, whereas the HF power reflects the degree of parasympathetic stimulation. Therefore, these measures can indicate emotional states associated with an autonomic arousal, such as anxiety, anger or fear.

7) If EOG or frontal or prefrontal EEG is recorded, the subject's EOG or (pre)frontal delta power associated with slow horizontal eye movements. As already explained, slow horizontal eye movements (“SEM”) are indicative of a transition from wakefulness to initial stage of sleep, and can therefore be used as one of the measures or indicators of drowsiness/alertness;

8) Variance of band-pass filtered EMG signal, if EMG is recorded. In fact, EMG does not have to be recorded with a special sensor/channel, since some amount of EMG from the facial and jawing muscles will be present in the EEG acquired from the scalp. High EMG activity is associated with active wakefulness, high mental engagement, or states of anxiety, fear, impatience, or anger. Therefore, EMG can be used in assessment of alertness, mental effort, workload, and emotional states;

9) Ratios of certain primary measures (from 1-3 and 7);

10) Ratios of each of primary measures 3-4 and 7 between different locations on the scalp;

11) Measures of time series interdependency such as covariance, correlation, coherence or mutual information (“MI”) between different signals, or different locations on the scalp. For an example, the subject's working memory is characterized by analyzing a distribution of the subject's workload measures (a complex function of the EEG PSD, described below) across multiple different scalp sites (Fz, Cz, PO, F3, F4, C3, C4, P3 and P4) during verbal and visuospatial memory tasks.

12) Weighted linear composites (e.g., linear combinations), or complex mathematical functions of the primary measures 1-11;

13) Differences between, ratios of, or complex mathematical functions of the primary measures obtained during baseline conditions requiring no mental effort versus the tasks/tests;

14) Differences between, ratios of, or complex mathematical functions of the primary measures between easy and more difficult task versions; and

15) Differences between, ratios of, or complex mathematical functions of the primary measures between initial and subsequent repetitions of the task in the same session.

In one embodiment, the primary measures, as well as the subject's demographic, anthropomorphic, and clinical data, and the information about concentrations or activities of relevant molecules from obtained biological samples are all fed in into module 90 that generates a variety of secondary scores that quantify the subject's cognitive functions and/or emotional states. Secondary measures include, for example, Drowsiness Level, Engagement, Mental Workload, Neurocognitive Factor Scores, Apnea Composite Evaluation Scores, Susceptibility to Sleep Deprivation, and Emotional State Index.

Module 90 includes a mathematical model or algorithm which will typically include a form of a discriminant function, although other available linear or non-linear multivariate statistical models can be used such as support vector machines, neural networks, hierarchical decision trees, and various forms of clustering algorithms. In an embodiment, module 90 uses the information about the emotional state of the subject to modify the scores describing cognitive functions.

For example, a score called “Engagement” is derived using a computer-implemented method described in U.S. Pat. Nos. 6,496,724 and 6,625,485, herein incorporated by reference, which relies on the acquisition of three baseline conditions and the prediction of a fourth mental state to determine the subject's alertness, mental effort and engagement during each of the sub-tasks by measuring on a second-by-second basis the power of the EEG recorded from multiple scalp locations at frequencies from 1 to 40 Hz in 1 Hz steps, and using a four-class quadratic discriminant function to convert these power measures into probabilities that the subject's level of mental effort and engagement belongs to one of the four classes defined as High Engagement, Low Engagement, Distraction and Drowsiness.

Similarly, a score called “Mental Workload” may be characterized using a two-class discriminant function to convert the power measures into probabilities that the subject is at Low or High workload.

In some embodiments, the levels (e.g., probabilities) of High or Low Engagement, High or Low Workload, Distraction and Drowsiness are analyzed across multiple timeframes to assess different aspects of the subject's cognitive and emotional state such as:

1) Over the entire session—shows the influence of drowsiness, level of interest, level of effort, stress and motivation over the test session including how these metrics changed over the session; or

2) During each of the sub-tasks to assess how each of the constructs in 1) were affected by each of the sub-tasks to identify particularly stressful or difficult sub-tasks;

To overcome the limitation of reliance on baseline data that may be biased as a result of the inability to acquire a truly representation of a required state (e.g., a disease or chronic condition may make it impossible to collect data in fully-rested condition); the present system includes a metric called the Linear Drowsiness Scale (“LDS”). The LDS is a continuous composite variable that represents a linear combination of the average reaction time during the last quartile of the 3-CVT and mean absolute and relative EEG power (FzPO and PO channels) during the 3-CVT and EC tasks at the following frequencies: 2, 3, 6, 7, 11, 15, 16, 17, 18, 21, 22, 23, 25, 26, 27, 28, 30 and 31 Hz. Generally, LDS is derived in three steps. In the first step, each of 50 primary variables used is normalized with respect to the existing normative database, for example, by performing a z-transform with mean and standard deviation for each variable taken from the database. In the second step, 10 intermediary composite variables are computed, for example: cr23—mean of the relative power at 2 and 3 Hz (delta range) from PO and FzPO channels from the 3CVT task; c78—mean of the relative power at 7 and 8 Hz (low alpha) from FzPO and PO channels from the 3CVT and EC task; c11—mean of the relative power at 11 Hz (high alpha) from FzPO and PO channels during the EC task; c2327—mean of the relative power at 23, 25, 27 and 27 Hz (low beta) from the PO channel and EC task; c3031—mean of the relative power at 30 and 31 Hz (high beta) from the PO channel and EC task; cd67—mean of the absolute EEG power at 6 and 7 Hz from the FzPO and PO channels during the 3CVT and EC tasks; cd9—mean of the absolute EEG power at 9 Hz from the FzPO and PO channels during the 3CVT task; ch1418—mean of the absolute EEG power at 21, 22 and 23 Hz from the FzPO and PO channels during the 3CVT task; ch2123—mean of the absolute EEG power at 21, 22 and 23 Hz from the FzPO channel and 3CVT task; and cs330—mean of the standard deviations of the relative power at 30 and 31 Hz during the 3CVT task in the third quartile (minutes 10-15). Finally, in the last step, mean value of the intermediate variables and all normalized primary variables are computed, taken logarithm of, and scaled by a factor of 10 to yield the LDS score.

In some embodiments, an alternative analysis is provided for the detection of a disease state based only on primary or secondary measures of behavioral performance, independent from electrophysiological, affective or other physiological measures or metrics are Neurocognitive Factor Scores (“NCFS”). The NCFSs preferably include four composite variables (factors): Visuo-spatial Processing Speed (“VSPS”), Sustained Attention (“SA”), Recognition Memory Accuracy (“RMA”) and Recognition Processing Speed (“RPS”). In an embodiment, the factors are derived from the measures of behavioral performance (e.g., mean reaction time, and % of correct responses) during the various tasks that are part of the AMP: Verbal Memory Scanning test, standard IR test, verbal PAL test, interference IR, numbers PAL, whole 3CVT and quartiles (0-5, 5-10, 10-15 and 15-20 min) of the 3CVT (a total of 20 primary measures). These measures are preferably organized in a 20×1 vector which is left-multiplied with a 4×20 matrix of factor score coefficients in order to yield 4 raw (non-normalized) factor scores. Each raw factor score may then be z-transformed with respect to the mean and standard deviation of the same score in a large reference population of normal subjects. VSPS, SA and RPS are finally multiplied with −1 in order to make their interpretation consistent with a common sense notion that negative values indicate poor performance whereas positive values indicate good performance. The resultant NCFS values are strongly associated with conventional clinical measures in a group of obstructive sleep apnea (OSA) patients undergoing CPAP treatment. Prior to treatment, RPS correlated strongly with Epworth Sleepiness Scale-ESS and Respiratory Disturbance Index—RDI; SA correlated with ESS and VSPS and RMA were associated with percentage of time arterial oxygen saturation—SpO2—was between 85 and 90%. Post-treatment VSPS and RMA were associated with residual hypoxemia and RPS with a combination of ESS, age and residual hypoxemia. Those skilled in the art will however recognize that similar models can be built for other diseases or states that dominantly affect a select group of the available behavioral measures. To build such a model one needs to record primary measures on representative samples of subjects with and without the disease or state, use the present system to derive available secondary measures, and develop a statistical classifier that would select optimal features out of the primary and secondary measures and compute probabilities of having or not having the disease or state. A multitude of the existing statistical classifiers (linear discriminant functions, support vector machines, neural networks, and so forth) can be used for this purpose. Therefore, the aforesaid NCFS should not be understood as the only model for predicting the disease or state available in the present system, and should by no means limit the scope of this invention.

In some embodiments, the present system also provides the capability to detect a disease state based only on primary and secondary measures derived from EEG, independent from behavioral, emotional or other physiological measures, called Apnea Composite Evaluation Scores (“ACES”). Scores such as ACES are preferably determined by module 90 and executed on computer 50. ACES are probabilities that a subject does or does not have sleep apnea (or more precisely, cognitive impairments due to sleep apnea). The model for ACES was developed on a group of 100 patients diagnosed with sleep apnea and 100 healthy controls and cross-validated on a different group of 95 healthy subjects and 97 OSA patients. The probabilities are calculated by means of two linear discriminant functions applied on the relative and absolute mean EEG power and standard deviations (“SD”) at selected frequencies from selected channels collected during selected tasks. Linear combinations computed (sums of products of each of the variables with the corresponding coefficient) are converted into probabilities in a standard way (assuming normal marginal distributions). Finally, the subject is assigned into NO OSA or OSA group based on which of the two probabilities is higher. Those skilled in the art will recognize that similar models can be built for other diseases or states that only or dominantly affect a select group of the available primary and secondary measures. To build such a model one needs to record primary measures on representative samples of subjects with and without the disease or state, use the present system to derive available secondary measures, and develop a statistical classifier that would select optimal features out of the primary and secondary measures and compute probabilities of having or not having the disease or state. A multitude of the existing statistical classifiers (linear discriminant functions, support vector machines, neural networks, and so forth) can be used for this purpose. Therefore, ACES should not be understood as the only model for predicting the disease or state available in the present system, and should by no means limit the scope of this invention.

For example, the score called “Susceptibility to Sleep Deprivation” can be derived by analyzing the time course of behavioral performance measures such as the reaction times and percentage of correct responses on the 3C-VT and PAL tests (see Experiment 1). Depending on the level of similarity (e.g., computed as a standard Mahalanobis distance) between the pattern of the subject, and patterns of the subjects from Experiment 1 that are stored in a database, the subject will be classified into one of the three categories: Low, Moderate or High susceptibility to sleep deprivation.

As stated above, the scores are preferably determined by module 90 and executed on computer 50. In one embodiment, the scores are designed so that they nominally provide “absolute” information, e.g., the subject is classified as either drowsy, or highly engaged, etc., and the LDS or the probability associated with high engagement can be taken as continuous measures of the “strength” of the made claim. However, in order to better account for the known high variability of any descriptors of cognitive functions or emotional states, significance of the derived scores can be further evaluated in one of the following ways:

1) Through a statistical comparison of the subject's scores on a particular test day or days with the normal range of variation of the subject's scores derived from the subject's earlier recordings obtained on a particular day or days;

2) Through a statistical comparison of the subject's scores on a particular test day or days with a typical normal range of variation of the same scores on the same tasks of a normative reference group of subjects having similar demographic characteristics and, if applicable, suffering from the same illness or other pathological condition that may impact cognitive functions or emotional state;

3) Through a statistical comparison of the subject's scores on a particular test day or days with data recorded on the subject just prior to exposing him or her to the battery of tests;

4) Through a statistical comparison of the subject's scores on a particular test day or days with the data recorded on the subject during exposure to a passive control condition;

5) Through a statistical comparison of the subject's scores on a particular test day or days with the subject's first recording;

6) Through a statistical comparison of the subject's scores on a particular test day or days with the subject's most recent recording;

7) Through a statistical comparison of the subject's scores on a particular test day or days with a mathematical combination of scores from the subject's prior recordings,

8) Through a statistical comparison of the subject's scores on a particular test day or days with scores from a specified subset of prior recordings from the subject; or

9) Through a statistical comparison of the subject's scores on a particular test day or days with a mathematical combination of scores from a specified subset of prior recordings from the normative reference group.

In all these data analysis examples, the phrase “statistical comparison” refers to an appropriate or applicable statistical test, such as t-test for dependent or independent samples, Z-test, repeated measures ANOVA, or between-subject ANOVA, and so forth. For example, paradigm (a) is best addressed by z-transforming the subject's scores on the day of interest using the mean and standard deviation of the subject's scores derived from the subject's earlier recordings, and then by evaluating the computed z-scores. Paradigm (b) is similarly best addressed by z-transforming the subject's scores on the day of interest using the mean and standard deviation of the same scores from the normative reference group, and then by evaluating the computed z-scores. Those skilled in the art will easily recognize what tests best address the paradigms (1)-(9).

A statistical model that differentiates between people who have and do not have a certain feature can generally be built for any disease, state or genetic trait that affects neurocognitive functions, affects or behavioral performance using the same methodology with which the NCFS or ACES were built. Such a model can subsequently be added to the existing pool of models. In order to build one, one may record available signals and/or measures on representative samples of subjects with and without the disease, state or trait, use the present system to derive available primary and/or secondary measures, and develop a statistical classifier that would select optimal features out of the primary and secondary measures and compute probabilities of having or not having the disease or state. Additional inputs to a model can include demographic information such as age, gender, pre- or post-menopausal, ethnicity, and so forth; anthropomorphic data such as weight, height, body mass index, neck or waist circumference, hip to waist ratio, and so forth; a medical history of diseases and current medications. A multitude of the existing statistical classifiers (linear discriminant functions, support vector machines, neural networks, and so forth) can be used for this purpose. By way of example, the present system may be used for detecting people at risk of developing hypertension, given that people with daytime somnolence or high susceptibility to sleep deprivation are known to be twice as likely to develop hypertension than people who are without somnolence and resistant to sleep deprivation. Therefore, ACES scores and LDS may be combined with demographic features (e.g., age, sex, BMI) and blood tests (e.g., lipids, cholesterol) in a statistical model that outputs probabilities of being at risk of developing hypertension. A similar approach may be used for developing scales and/or metrics other than the LDS, Engagement, Workload, and so forth. For example, in order to make a metrics that operates on second-by-second physiological data and classify each second into one of N classes, one can collect relevant physiological data on a representative set of subjects, and have experts score each second of the data according to some existing rules or their expert knowledge. A linear or non-linear classifier is then be trained on this set, and validated on a different set of subjects from whom the same physiological signals have been obtained.

### Database and Profile Module:

In one embodiment, the scores derived by module 90 are subsequently stored into database 120 for later off-line analyses. An individualized, detailed report called the “Psychophysiological Profile” of the current and comparative (e.g., either to self at baseline or a population of relevance) cognitive function will be generated and also stored in the database 120. In one embodiment, the psychophysiological profile is generated by profile module 100. If the subject 10 is at any point diagnosed with a disease for which a normative group of records exists in the database 120, the record of this subject 10 may be added to that normative group. In this way the normative database will continuously grow, which will in turn increase the accuracy and discriminatory power of the present system.

In addition, in some embodiments, the database 120 accumulates data of each examined subject 10 over time, and combines them into a single report (which may also be part of the psychophysiological profile) that allows a user of the system to see changes over time. The user is able to determine what aspects of cognitive function he would like to see, and can choose the comparison data. The user can examine data in the database either locally or over the Internet.

In one embodiment, the psychophysiological profile generated by profile module 100 includes 1) demographic, anthropometric, clinical, or other data pertaining to relatively constant or slowly changing (e.g., over weeks, months or years) traits and characteristics of the subject; 2) various afore described primary and secondary measures derived from the behavioral performance and/or recorded physiological signals; 3) data related to concentrations and/or activities of various bio-markers; or 4) any combination thereof. In one embodiment, the Profile system may relate to the information gathered at only one point in time (e.g., on a single day), or it may present a time course of the relevant measures. Different ways of presenting the data can be selected for different data types; for example, demographic data can be presented in a form of a table, whereas most secondary measures such as ACES, LDS, or probabilities of High or Low engagement can be presented graphically, together with the normative values for the same measures derived from the database.

Yet, in another embodiment, the profile module 100 can integrate the information contained in the data listed in 1)-4), and include suggestions of what possible diseases, states or genetic traits could have caused the observed pattern of neurocognitive, behavioral and physiological measures, as well as provide recommendations about available interventions or treatment options. For example, subjects that are suspect of having OSA can be separated into several clinically relevant categories (e.g., mild, moderate and severe disease with or without daytime sleepiness, and with or without significant cardio-vascular involvement) based on the measures of daytime sleepiness (e.g., LDS), performance on neurocognitive tests (e.g., clinically relevant information would be whether a memory deficit exists or not, whether the LF power of the HRV signal that reflects the degree of sympathetic activity, is low, moderate or high), relevant bio-markers (e.g., serum lipids) and demographic and clinical data (e.g., age, sex, body-mass index, cardio-vascular comorbidities, etc.). In some embodiments, the generated psychophysiological profile includes statements about estimated severity of the disease, detected cardiovascular irregularities (e.g., high variability of heart rate detected from the EKG that may suggest arrhythmias, or high LF power that may be correlated with high blood pressure), and recommend further examinations (e.g., blood pressure check-up at a physician's for subjects with detected cardiac irregularities, an overnight sleep study for subjects with high LDS and ACES scores) or enlist available treatment options (e.g., CPAP machine vs. oral appliance).

Additional examples of pieces of information that may be presented in a report can be found in sections of this document presenting different experiments and/or validation studies done with the present profile module.

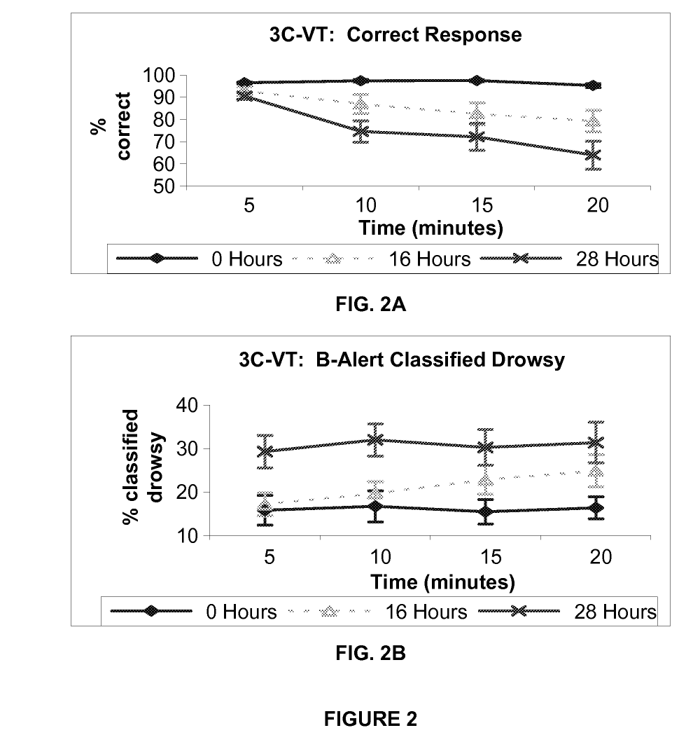

Turning now to FIG. 2, the time course of one behavioral measure (% of correct answers) is shown in FIG. 2A and the B-Alert drowsiness classifications (measured according to U.S. Pat. Nos. 6,496,724 and 6,625,485) during 20 minutes of 3C-VT test is shown in FIG. 2B. Deterioration of performance and/or changes in EEG or other physiological metrics over such a short time span may be indicative of traits or states such as high susceptibility to sleep deprivation, or inability to sustain attention that may be associated with stroke or traumatic brain injury. On the other hand, in depression performance will be constantly lower than normal, whereas the B-Alert drowsiness classifications, the LDS or other EEG indices may remain unchanged. In one embodiment, the generated psychophysiological profile, in addition to simply presenting the time course of the drowsiness classification during the cognitive test, includes suggestions of what the cause of the observed result might be and what further steps can or should be taken with regard to that (e.g., “Slow reaction times coupled with insignificantly lowered performance measures, normal LDS and ACES in middle-aged women can be suggestive of depression. A referral to a specialist is recommendable” or “Progressively deteriorating performance indices, lengthening reactions times, high probabilities of being classified as Drowsy, high LDS scores, normal ACES and high LF/HF HRV ratio in younger men are indicative of an acute sleep deprivation”).

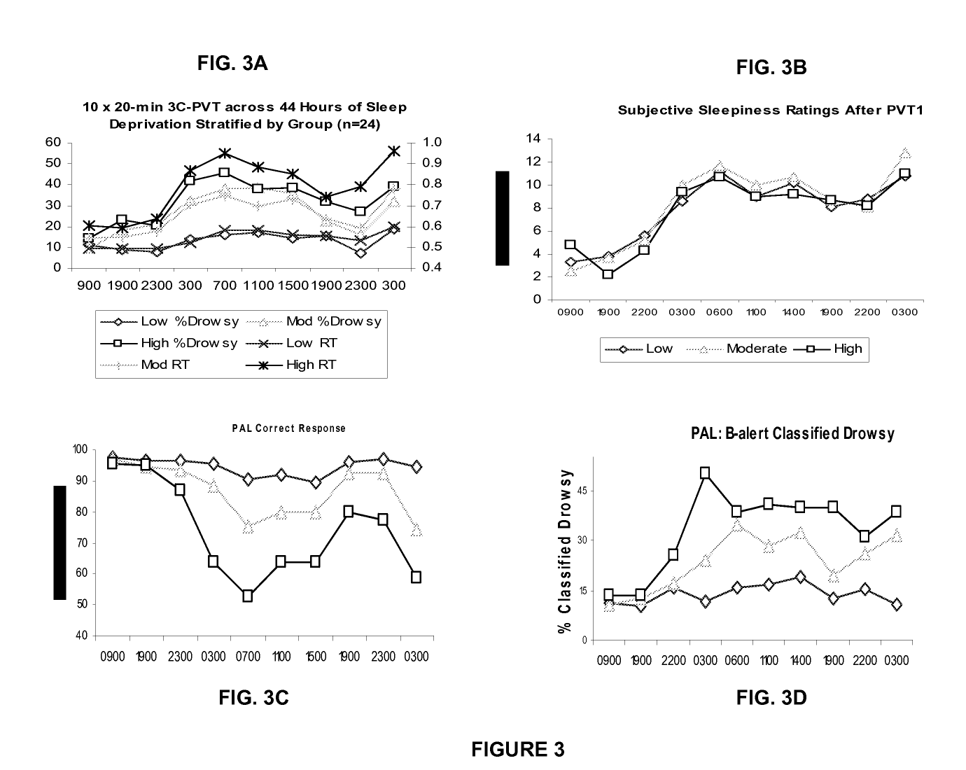

The generated psychophysiological profile can also show changes in primary and secondary measures over longer periods of time. Turning to FIG. 3, the time course of B-Alert EEG drowsiness classifications over the 44-hours and average reaction times during each of the ten performances of the 3C-VT is shown in FIG. 3A for each of the three sleep deprivation vulnerability groups (High, Moderate and Low susceptibility to the effects of sleep deprivation). FIG. 3B shows the self-reported sleepiness levels for each of the three sleep deprivation vulnerability groups at each of the ten test points. (Note that there were no differences between the groups in their self-reported sleepiness, confirming that people are generally unaware of how sleepy they are and how much that is impairing their performance.) FIGS. 3C and 3D show the accuracy of performing a simple memory test and the EEG drowsy classifications during the memory test at each of the ten time points and for each of the three sleep deprivation vulnerability groups.

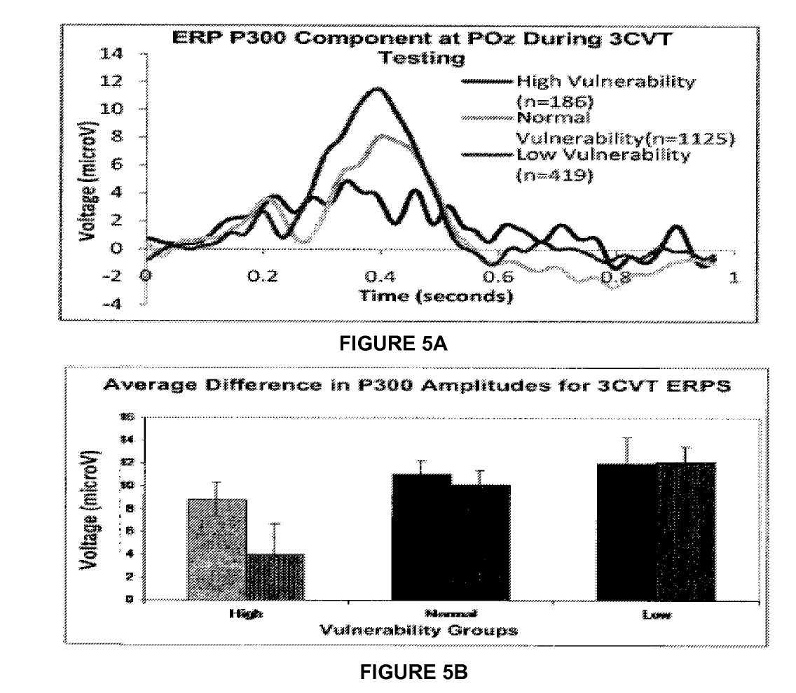

Turning now to FIG. 5, changes in event-related potentials caused by sleep deprivation over a 4-week period is shown. Both FIGS. 5A and 5B also indicate that the person tested will be classified into one of the three groups: high, normal and low susceptibility to sleep deprivation. Assessments of susceptibility to sleep deprivation are important for professions such as truck drivers or pilots. In one embodiment, the generated psychophysiological profile includes warnings and recommendations for further evaluation procedures (e.g., “the driver shows very high susceptibility to sleep deprivation—management intervention may be required” or “significant daytime sleepiness accompanied with slower reaction times and some memory deficits detected—the subject should be evaluated in an overnight sleep study”).

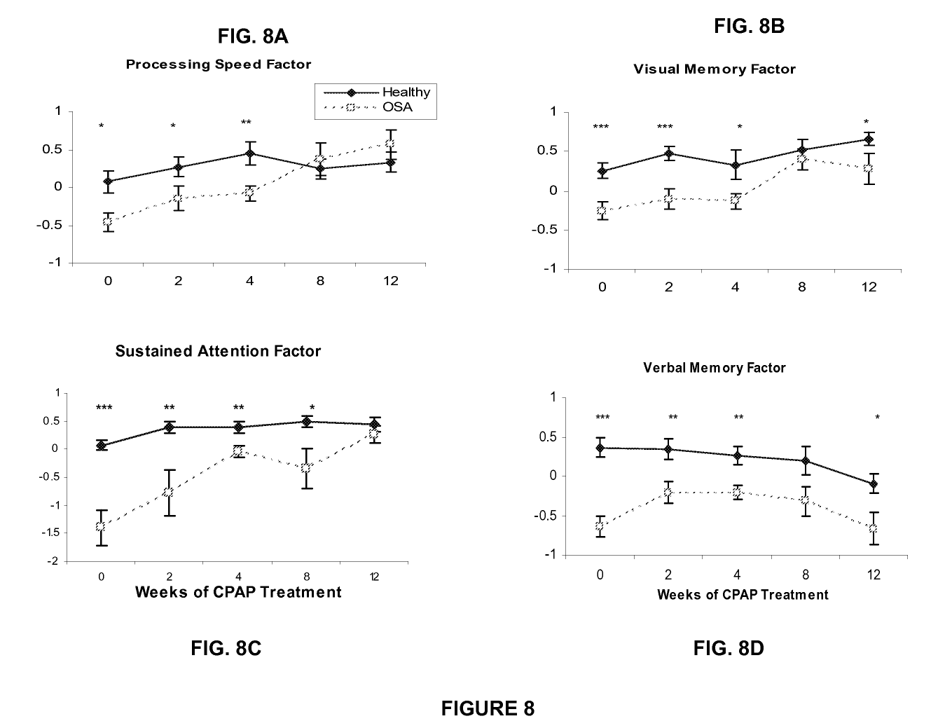

An example of a treatment-induced change in measures of neurocognitive functions is provided in FIG. 8, where a drop in the ACES is observed after several weeks of treating obstructive sleep apnea with a CPAP machine. Such information will be needed for a clinician to evaluate the effect of a prescribed treatment, and determine whether other treatment options (e.g., oral appliance or surgery) should be considered.

Other examples of suggestions about possible treatment options provided in the generated psychophysiological profile may include: recommending an attention training with neurofeedback therapy to a subject with low scores on sustained attention task, recommending a relaxation training to a subject with high anxiety measures, or suggesting the use of omega 3-fatty acids in the chronically sleep-deprived subjects.

### Use of the Profile Module:

Some models of detection of a disease have been mentioned when the NCFs or ACES have been discussed. An example of a more complex model of detection of a disease is differentiation between depression and obstructive sleep apnea in women, since both conditions can clinically present with complaints suggesting insufficient sleep quality, daytime somnolence and impaired neurocognitive functions. The model inputs may include demographic variables such as age and menopausal status; anthropomorphic variables such as weight, height, BMI and neck circumference; information about co-morbidities such as diabetes, hypertension, stroke; information about behavioral signs of OSA such as snoring; blood tests such as lipid levels or hormones; performance measures on cognitive testing; and primary and secondary physiological measures (derived from EEG, EOG, EKG or any other acquired signal). In one embodiment, the model outputs three probabilities: 1) having depression, 2) having OSA, and 3) not having either disease.

Preliminary evidence reveals a number of commonalities between depressed and OSA patients in the neurocognitive profile and in fact it is not uncommon for OSA to be misdiagnosed as depression by primary care and other treatment providers. OSA and depression results in slower processing speed, verbal memory impairment and self-reported fatigue. However, Heart Rate variability (HRV) in depression is frequently suppressed and this symptom reportedly reverses following successful treatment. In contrast, OSA patients generally exhibit sympathetic overload (as measured by increased low frequency in the PSD for HRV). Thus, the addition of the HRV parameter may be the key to distinguishing these disease states and to providing the correct diagnosis and treatment. As the technology evolves, it is anticipated that a more definitive multi-parameter statistical model (similar to the ACES model) will be developed for distinguishing many disease states. Such a multi-parameter statistical model will rely on multiple disassociated measures, thereby allowing for an improved differential diagnosis.

Some examples of how a progression of, or recovery from, a disease or injury may include:

E1) Monitoring the time course of changes in NCFs, measures of distractibility, and other aforesaid secondary scores in patients who have suffered a traumatic brain injury. A gradual recovery is expected in general over the first year following the injury; however, different functions may be recovering at different pace, and monitoring the dynamics of NCFs or other provided secondary measures may help identify functions whose recovery is not satisfactory (e.g., long-term memory may recover, but short term not). In some embodiments, the profile module 100 includes the time courses of various scores such as NCFs and also provides the information about what the observed pattern of changes over time may mean (e.g., a progressive rapid recovery, progressive but gradual recovery, initial rapid recovery that has reached a plateau, visit-to-visit fluctuations without any real change, deterioration, and so forth). Appropriate therapeutic interventions could subsequently be undertaken.

E2) Monitoring the time course of changes in the aforesaid secondary scores as well as of concentrations of biological molecules such as IL-1, IL-6 or TNF in children with attention-deficit-hyperactivity-disorder (“ADHD”) or mild autistic spectrum disorder (“ASD”). The assessment is in this example complicated by the fact that a normal progression/maturation of cognitive functions that occurs with age may be taking place concurrently with the regression caused by the disease/disorder, and with improvements related to the applied therapeutic procedures.

Some examples of evaluating the effect of treatment on a disease may include:

E3) Evaluating the change in ACES, LF power and other aforesaid scores in patients with obstructive sleep apnea following the treatment with CPAP device. Typically, a significant drop in the ACES score and LF power, and increases in almost all other scores are seen as compared to the pre-treatment value, indicating less daytime sleepiness, less sympathetic activity, and improvements in other cognitive functions (See Experiments 3 and 4). In one embodiment, the generated psychophysiological profile includes recommendations for further evaluation or treatment options in addition to the aforesaid measures/scores. For example, if the ACES, NCFs and HRV measures became normal or nearly normal following the use of CPAP, the generated psychophysiological profile would advise continuation of the therapy. However, if residual daytime sleepiness is detected on the LDS, the profile may enlist possible reasons (e.g., poor adherence to the CPAP therapy), suggest further evaluations if the adherence has not been the reason (e.g., overnight sleep study), or recommend other evaluative procedures or treatment options (e.g., oral appliances for subjects with dominantly positional OSA).

E4) Evaluating the change in ACES, LF power and other aforesaid scores in patients with obstructive sleep apnea following the treatment with oral appliance. A significant drop in the ACES score and LF power, and increases in almost all other scores except for those describing long-term memory are seen as compared to the pre-treatment value, indicating less daytime sleepiness, less sympathetic activity, and improvements in other cognitive functions (See Experiment 4).

### Validation of the System:

In what follows, examples are provided which show how the disclosed analytic techniques can be applied or combined to measure cognitive function or changes in neurocognitive function in healthy individuals and clinically significant populations. These analyses or similar applications of the system can be combined to provide or derive psychophysiological profiles for profile module 100.

### Experiment 1: Predicting Susceptibility to Sleep Deprivation

Introduction:

An a-priori identification of individuals prone to mental errors or susceptible to fatigue as a result of sleep deprivation could improve safety and productivity.

Research Protocols:

Twenty-four healthy subjects (males=18, females=6; mean age=24.9; range 21-38) completed a baseline Alertness and Memory Profile (AMP) session (beginning ˜0900 on Friday) and nine AMP session beginning ˜1900 Friday and concluding ˜0500 on Sunday. A 1-hour break was provided between each AMP s between each session; a 40-minute nap was provided at 1900 on Saturday. Technician-observed drowsiness (available for 18/24 subjects), reaction times (“RT”), and percent correct responses were computed across subjects for each AMP battery. Within-subject correlations across batteries were computed.

Results:

Repeated measures ANOVA across the 10 time-points revealed progressively increasing drowsiness in all indices (B-Alert classifications, technician observations, and performance) during 3C-VT and IR (all p's<0.001). The graphs in FIG. 3 illustrate the similar patterns in the effects of sleep deprivation across measures including the EEG classifications, 3C-VT reaction time and accuracy, IR accuracy and technician-observed drowsiness. To validate the EEG metrics, Pearson product-moment correlations between B-Alert EEG classifications and the 3C-VT reaction time and between EEG and technician observations were computed for each individual subject.

Performance and B-Alert data from the 3C-VT data were analyzed in four 5-minute blocks from three time points: 0 hours (baseline), 16 hours and 28 hours (See FIG. 2). A 3 (session)×4 (time) repeated measures ANOVA revealed significant main effects for session (F=9.69, p<0.001) and time (F=28.68, p<0.001) for percent correct responses and a significant Session×Time interaction (F=8.59, p<0.001) and significant main effects for session (F=5.24, p<0.01) and time (F=3.07, p<0.05) for the percentage of drowsy EEG suggesting that both measures are sensitive in identifying individuals with excessive sleepiness as a function of time-on-task.

Results-Individual Differences:

Thresholds were applied across time-points to the 3C-PVT, IR and MWT performance measures to stratify individuals into three groups (low, moderate, or high vulnerability) based on vulnerability to sleep deprivation. A 3(Group)×10(Time-points) repeated measures ANOVA revealed significant main effects for group for B-Alert % Drowsy (F=7.23, p<0.01), 3C-PVT reaction time (F=33.6, p<0.001), and 3C-PVT % correct (F=31.0, p<0.001). A main effect for group for B-Alert % Drowsy (F=3.95, p<0.05) was also obtained during the IR. Group×Time interactions for these variables were also significant at the p<0.01 level (See FIG. 3). Interestingly, the Subjective Sleepiness Scale did not reveal significant group differences. Pair-wise comparisons between groups showed that the RT and B-Alert % Drowsy discriminated Low and Moderate groups from the High group (p<0.01) beginning at 1900 (See FIG. 3A), provide measures for a “biobehavioral assay” to identify individuals most susceptible to sleep deprivation.

Conclusions:

The baseline AMP was capable of deriving information needed to normalize the EEG to generate accurate B-Alert EEG classifications. Neurocognitive measures (e.g., EEG, performance, EKG, etc.) can be further profiled in longer duration tasks by dividing the testing into segments and comparing the segment results within- and between-subjects to a database of measures. Repeated-measures AMP sessions provided neurocognitive indices capable of differentiating group characteristics. The B-Alert classifications were highly correlated with technician observations, visual inspection of the EEG, performance measures of alertness and subjective sleepiness. The combination of the B-Alert classifications and neuro-behavioral measures at multiple and distinct time points were able to identify individuals whose performance is most susceptible to sleep deprivation. Of interest, the subjective measures of sleepiness did not distinguish between the groups, suggests that the subjects' perception of their own level of fatigue did not correspond to the objective measures.

The rate of decline in the subject's ability to sustain attention during the 3C-VT can be measured by calculating the slope of the least-squares regression line across quartiles for the averages of the following variables: percent correct, reaction time, and percent EEG epochs classified as drowsy.

### Experiment 2: Psychophysiological Profiles of Sleep Deprivation and Stress

Introduction:

Beyond sleep deprivation, individuals (e.g., combat soldiers, factory workers, etc.) may be exposed to other factors which can contribute to dangerous consequences, including but not limited to stress and fatigue. This study evaluated the change in neurocognitive measures as compared to psychophysiological markers of stress and fatigue.

Methods:

USMC battalion/platoon leaders (n=17) were evaluated during 28-day, live-fire training exercises with continuous actigraphy and a baseline and weekly AMP session using an abbreviated montage of EEG and EKG acquired during a 3-Choice-Vigilance-Test (3C-VT). Self-reported stress, fatigue and mood were assessed with Profile of Mood States, Stanford/Karolinska sleepiness scales, Brief Fatigue Inventory and Perceived Stress Scale.

Results:

The baseline AMP session was used to accommodate individual differences in the EEG in order to accurately generate patented B-Alert neurocognitive measures (e.g., High-Engagement, Low-Engagement, Drowsy or Sleep Onset) at one-second increments. The total percentage of epochs in each class was calculated for each 5-minute quartile of the 3C-VT. Reaction-time (“RT”), percent-correct and percent-missed (lapses) were computed by 3C-VT quartile. A 4(Week)×4(Quartile) RMANOVA revealed significant interaction effects (p<0.0001) across quartiles over time in the 3C-VT for all EEG classes with increasing Drowsiness and decreasing High-Engagement across weeks of training accompanied by increasingly impaired 3C-VT performance. HR decreased significantly (p<0.0001) across weeks of training.

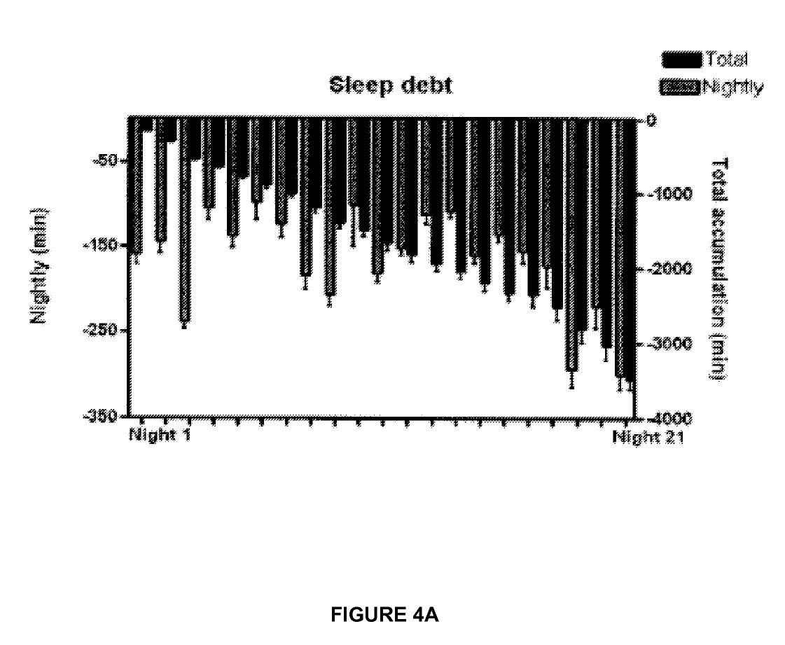

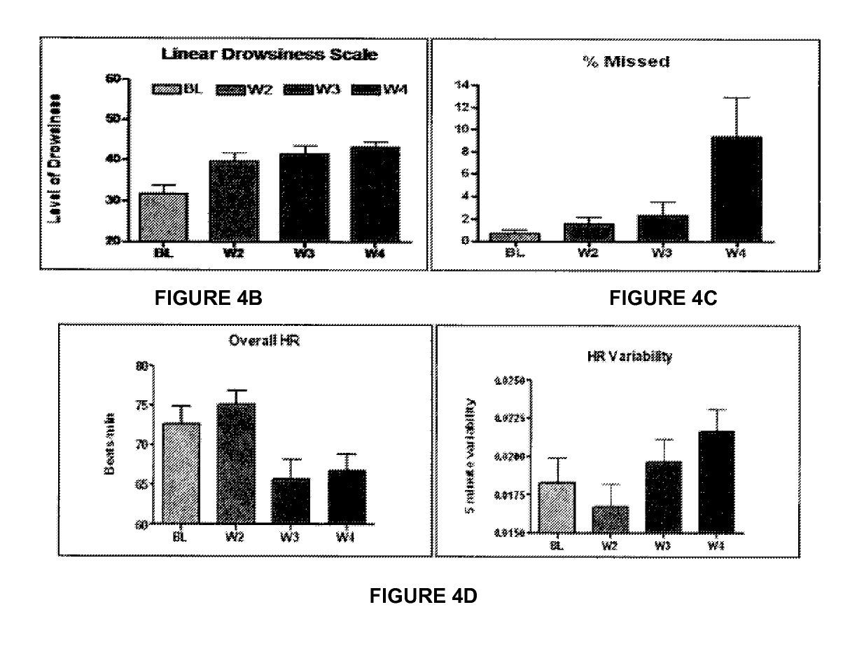

Panel A of the FIG. 4 shows that the trainees had some degree of sleep debt (actual minus recommended, 8 h) each night (orange bars), with a significant amount of sleep debt accumulating over the training course (blue bars). An EEG-based linear drowsiness revealed significant increases in drowsiness over the course of the training panel B, anchoring data from healthy subjects at baseline, 28 h and 33 h sleep deprived). The LDS scores of the marines after the 21 days of training are equivalent to those of healthy individuals deprived of sleep for 28 h, indicating a severe level of fatigue. In addition, the LDS revealed that some Marines arrived for Mohave Viper training significantly fatigued (4/8 in July, and 1/7 in August battalions). As a result the investigators were unable to acquire fully-rested baseline data from the Marines participating in this study.

Errors (% missed) increased on the 3C-VT as sleep debt accumulated over the course of training as well (panel C). Although not significant, there was also a trend toward increased reaction time over the 21-day training. The final two graphs show that HR fell over the training course, while HRV increased (panel D). Increased HRV is thought to be associated with increased stress and decreases in HR have been reported to occur as a result of sleep deprivation.

In addition to the B-Alert EEG indices, Event-Related Potentials (“ERPs”) were calculated by time-locking the EEG to the onset of the 3C-VT stimuli and averaging for all of the correct targets identified during the 3C-VT. The target-related P300 component was measured for each individual's grand means acquired during week one and week four. The P300s were calculated by visually inspecting the ERP graphs and determining the peak to trough amplitude values, as well as latencies at those values. Peaks were only used if they were between 275 and 600 ms post-stimulus and troughs between 150 and 300 ms post-stimulus (See FIG. 4).

Cluster analysis was applied to stratify the Marines into 3 vulnerability groups based on performance metrics, and the amplitudes of the P300s were compared for the three groups (See FIG. 5A). Weeks 1 and 4 for the High Vulnerability group were significantly different. Week 4 for High Vulnerability was also significantly different from Week 4 for both the Normal and Low Vulnerability groups (See FIG. 5B). These data indicate that EEG and HR measures were able to assess the fatigue indicated by the significantly increased errors and inattention (p<0.0001) found during 3C-VT. The use of the ERP P300 component acquired during the completion of the 3C-VT offers another potential biomarker for identifying variability across individuals.

Conclusions:

Comparing baseline measures to a database of similar measures provided an initial assessment of individuals at risk of stress and fatigue. Repeated measures testing shows changes in multiple measures which can be combined to identify groups with distinct vulnerability levels.

Experiment 3: Unique Neurocognitive Measures Associated with Disease States

Introduction:

Obstructive Sleep Apnea (“OSA”) is the most common disorder observed in the practice of sleep medicine and is responsible for more mortality and morbidity than any other sleep disorder. OSA is characterized by recurrent failures to breathe adequately during sleep (termed apneas or hypopneas), usually due to collapse of the upper airway. These events can cause repetitive hypoxemic episodes and sleep fragmentation. OSA causes daytime drowsiness and cognitive deficits and increases risk for hypertension, congestive heart failure, coronary artery disease, myocardial infarction, cardiac arrhythmias, stroke, diabetes and depression. The severity of OSA defined by the Respiratory Disturbance Index (“RDI”, the average number of abnormal breathing events/hour of sleep) can be evaluated with overnight polysomnography (“PSG”) in a sleep laboratory, or with in-home techniques, however, the assessment and quantification EDS and the cognitive impairments resulting from OSA, has proven more difficult

Methods:

Healthy controls and OSA patients were evaluated with an AMP including 5-minute eyes open and 5-minutes of eyes closed with paced button press, a 3C-VT, Image Recognition Tests with and without Interference, Verbal/Image PAL tests, and Sternberg VMS. All subjects were studied at baseline, and 2, 4, 8 and 12 weeks subsequent to baseline with the OSA patients being treated with CPAP.

TABLE 2
Healthy and OSA patient sample size for each study.

| | Baseline | 2 weeks | 4 weeks | 8 weeks | 12 weeks |
|---|---|---|---|---|---|
| Healthy | 61 | 56 | 55 | 46 | 45 |
| OSA - up to 5 time | 62 | 51 | 50 | 20 | 21 |
| points | | | | | |
| OSA - 2 time points | 100 | 0 | 86 | 0 | 0 |
| (Validation) | | | | | |

Continuous EEG was acquired during the AMP using a wireless sensor headset and automated B-Alert software. Performance variables derived from the 3C-VT, VMS, Standard, Verbal, IR Interference and Numbers PAL tests included: % correct, % incorrect, % missed and reaction time (“RT”). For the 20-minute 3C-VT, % correct and RT were also calculated for each 5-minute period. Slope values were calculated for change over 5-minute quartiles of the 3C-VT (% correct quartile slope, RT quartile slope). The EEG variables derived during the AMP tests were based on absolute and relative Power Spectral Density (“PSD”) values calculated for FzPOz and CzPOz bipolar recordings. Absolute EEG variables were the logged PSD values after artifact decontamination for each one-Hz bin from 1 Hz to 40 Hz. Relative power was also computed by taking the ratio of the logged PSD value and the total power from 3 to 40 Hz for each task.

Results: Between-Group Comparisons for AMP Performance:

The OSA patients, as a group, showed decreased 3C-VT accuracy with more lapses and slower RT when compared to the healthy group at the 2-week and 4-week post-treatment sessions. After 8 weeks of CPAP treatment, RT and lapses were no longer significantly different between the two groups. After 12 weeks on CPAP, all measures derived from the vigilance test for the OSA patient population were comparable to those of the healthy subjects. The amelioration of the learning and memory deficits observed following CPAP treatment followed a different pattern and time course. OSA patients evidenced significant impairment in both verbal and image learning and memory in comparison to the healthy subjects, even after 12 weeks on CPAP. These results are comparable to previous reports that cognitive deficits in OSA patients may only be partially reversible with CPAP therapy.

Results: AMP Composite Evaluation Score (ACES):

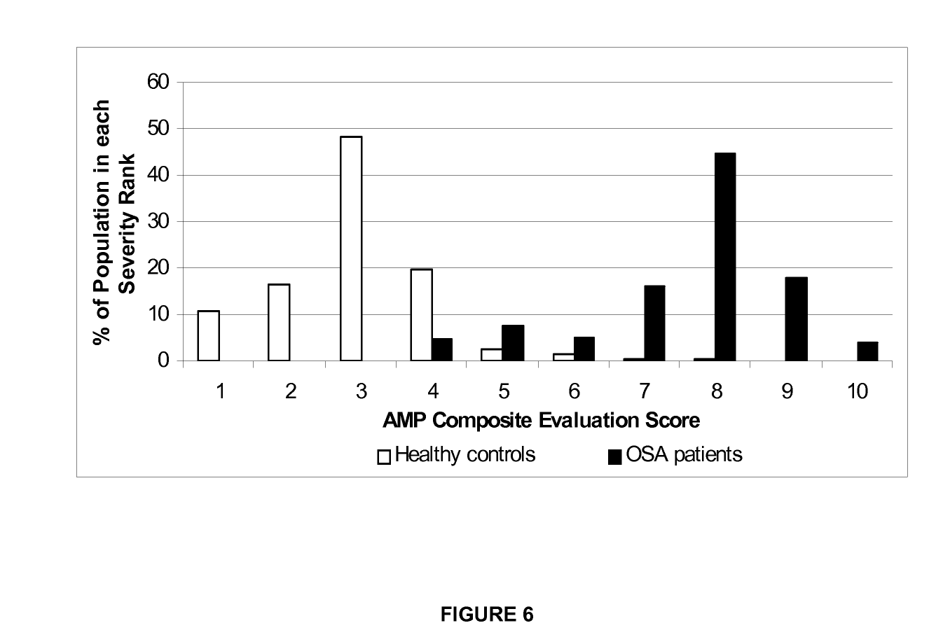

A two-class discriminant function analysis (“DFA”) was performed using a combination of EEG and performance measures from 195 healthy controls and 197 OSA patients prior to CPAP treatment (mean RDI=51.2; range 5 to 165). Half of the healthy subjects and OSA patients were randomly selected to develop the DFA model, and the remaining half were used to cross-validate the model. Sixty variables were selected to optimize discrimination between the OSA and healthy groups using stepwise multiple regression analysis. Only 2 of the 60 variables selected by the DFA were based on performance during the 3C-VT: % correct third quartile, and RT fourth quartile. No performance variables from the IR were selected. The majority of variables selected were derived from the EEG including measures of the variability in EEG within a particular task. For each subject, the DFA outputs were used to calculate the probability that the subject suffered from OSA. This probability result was used to calculate an AMP Composite Evaluation Score (ACES) ranging from 1 (healthy) to 10 (severe OSA). The DFA model accurately identified fully-rested healthy subjects and pre-treatment OSA patients, providing a sensitivity of 0.88 and a specificity of 0.96. After ACES was calculated using probabilities from the discriminant function model, 83% of OSA patients had a score of 7 or greater and 95% of the healthy subjects had a score of 4 or less. The distribution of ACES for OSA and healthy subjects is presented in FIG. 6.

ACES Post-Treatment with CPAP:

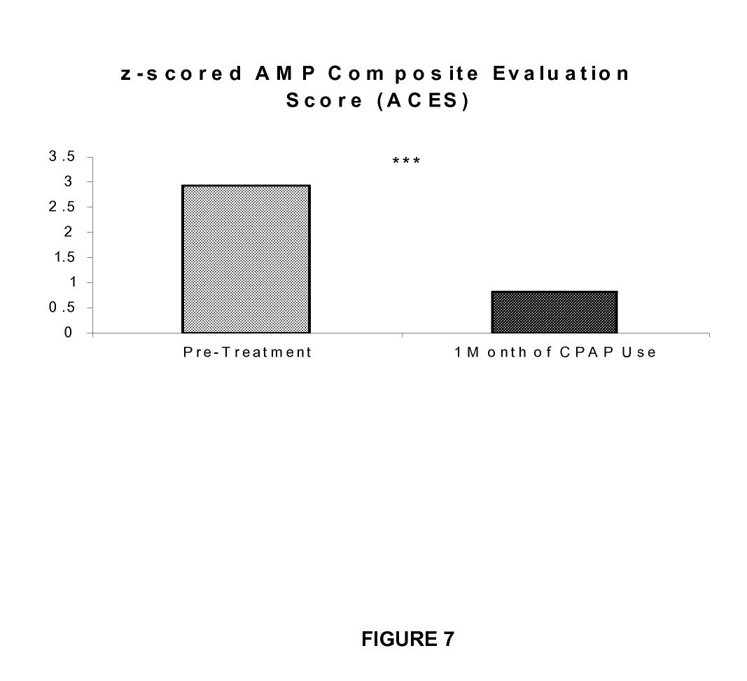

After one month of CPAP treatment, the 167 OSA patients exhibited significant improvements in most objective measures. The ACES metric was z-scored to the healthy population. The change in the Z-scored ACES after one month of CPAP indicated that the majority of OSA patients moved significantly towards the healthy control subjects ACES values (See FIG. 7). Although the changes in ACES after one-month of treatment suggests a group trend towards the healthy population, there were a significant number of OSA patients that did not evidence progress as measured by ACES. This may reflect the presence of residual neurocognitive impairments, the effects of chronic hypoxemia or may be evidence of genetic or trait characteristics associated with OSA. Further delineation of these influences on the ACES are currently under investigation. An ideal metric would provide differentiation of genetic factors (trait) that distinguish OSA patients from healthy controls as well as distinguish state variables including alertness/memory impairments. Further research is required to determine whether these influences on the ACES score can be separated.

Results: Factor Analysis of the AMP Performance Data:

One of the goals of the study was to determine whether a differential quantification of alertness and memory could be achieved. All performance measures were submitted to a factor analysis to assess whether a summary set of latent performance domains could be derived within the data. The following variables for 3C-VT, Verbal Memory Scanning, Standard IR, Verbal PAL, Interference IR, and Numbers PAL were included in the initial set: % correct, % incorrect (but not % missed), and RT. In addition, % correct quartile slope and RT quartile slope for change over five-minute segments of the 3C-VT were added to the set.

The final iteration of the factor analysis resulted in four factors that were labeled according to the pattern of factor loadings. Simple structure was largely achieved with all variables having a primary loading of greater than 0.6 and secondary loadings of less than 0.4. Calculated factor scores were reversed as necessary so that positive scores indicated better performance: Processing speed: all RT measures, Visuospatial memory: Standard, Interference and Numbers PAL % correct and % incorrect, Sustained attention: 3C-VT % correct, % incorrect, 3C-VT quartile and slope. Verbal memory: Verbal Memory Scanning, Verbal PAL % correct, % incorrect. The calculated factor scores are presented in FIG. 8. Processing speed (FIG. 8A) and sustained attention (FIG. 8C), which are associated with alertness, showed significant improvement with CPAP. Results for visual memory (FIG. 8B) and verbal memory (FIG. 8D) were more difficult to interpret possibly due to greater variability, training effects, and small sample sizes after 8 and 12 weeks of CPAP. Verbal memory improved significantly after 2 weeks of CPAP, but showed no further improvement with treatment. Similar research studies suggest that higher cognitive functional impairments may be only partially reversible with CPAP

Results: Relationship Between Neurocognitive Factor Scores and Diagnostic PSG Data:

An analysis was conducted with the same group of patients to examine the relationships between the PSG data including the RDI, hypoxemia levels and the neurocognitive impairments prior to and following treatment with CPAP. The goal was to begin to address the controversy over the extent to which the cognitive impairments in OSA are attributable to sleepiness or hypoxemia and to assess the reversibility of these impairments following treatment. Stepwise multiple regression examined the relative importance of RDI, hypoxemia, Epworth and age on the Neurocognitive Factors: processing speed (“PS”), visuospatial memory (“VSM”), sustained attention (“SA”) and verbal memory (“VM”). factors. Prior to treatment, RDI/Epworth predicted PS (R=0.336), Epworth predicted SA (R=0.225) and SpO2 85-90% predicted VSM (R=0.225). Post-treatment VM was predicted by hypoxemia SpO280-85% (R=0.309) and PS by a combination of Epworth/Age/SpO2<80% (R=0.440).

These data suggest that hypoxemia is closely related to learning and memory impairment pre-treatment while alertness factors (e.g., PS, SA) were predicted by subjective sleepiness (Epworth). Surprisingly, RDI related only to PS. Post-treatment, hypoxemia was an important determinant of the residual neurocognitive impairments observed in VM. The findings support the theory that the various manifestations of OSA differentially affect neurocognitive functioning and that some impairments persist even after successful amelioration of sleep-disordered breathing.

Conclusions:

Two methods for quantifying the results were developed based on this initial data set: the “AMP Composite Evaluation Score,” or “ACES” and the “Neurocognitive Factor Scores”. The ACES can be derived using just the brief-AMP protocols and the neurocognitive factor analysis requires the full AMP session. The ACES proved highly sensitive and specific for discriminating untreated OSA patients from healthy controls. The ACES was successfully able to discriminate healthy subjects from OSA patients with a sensitivity of 88% and a specificity of 95% using both EEG and performance measures.

### Experiment 4: Repeated Measures Assessment of Changes in Neurocognitive Measures

Introduction:

Obstructive Sleep Apnea (“OSA”) causes impairments in neurocognitive function including deficits in attention, learning and memory and slower processing speed and alertness. Treatments for OSA such as Mandibular Repositioning Devices (“MRDs”) and continuous positive airway pressure (“CPAP”) should contribute to the amelioration of these symptoms.

Methods:

Three groups were studied, including healthy controls (n=46), OSA patients treated with MRD therapy (n=30, mean RDI=18+10.2, range 5-54) and OSA patients treated with CPAP (n=30, mean RDI=23+10.7, range 5-39). The three groups were studied at an initial baseline and again approximately 30 days later (representing a one-month treatment intervention for the MRD and CPAP groups).

A sub-set of tasks obtained with the AMP were used including a 3-Choice-Vigilance-Test (“3C-VT”), Image-Recognition (“IR”), IR with Interference (“I-IR”), Verbal/Number-Image Paired-Associate-Learning (“VPAL”/“NUM-PAL”), and Sternberg-Verbal-Memory-Scan (“VMS”). Neurocognitive function scores were compared to subjectively reported changes in sleepiness (Epworth Sleepiness Scale—“ESS”) and depression (Beck Depression Inventory—“BDI”).

Results:

3(group)×2(time) RMANOVA showed pre-treatment OSA patients in MAD and CPAP groups had significantly slower reaction times (“RT”) and decreased accuracy (% correct) on 3C-VT (F=15.25, p<0.0001; F=18.96, p<0.0001), VMS (F=40.62, p<0.0001; F=17.73, p<0.0001), IR (F=8.43, p<0.01; F=4.66, p<0.05) and I-IR(F=12.67, p<0.001; F=26.64, p<0.0001) and showed higher risk for OSA in an EEG composite evaluation score (F=19.92, p<0.0001) compared to healthy controls at both baseline and 1-month. Both MAD and CPAP groups evidenced significantly faster RT and increased % correct on 3C-VT(F=13.10, p<0.001; F=13.79, p<0.0001), VMS(F=22.10, p<0.0001; F=8.93, p<0.01) and IR-I(F=5.12, p<0.05; F=19.32, p<0.0001) and improved EEG scores (F=42.06, p<0.0001) after 1-month of therapy. BDI and Epworth scores were significantly decreased after 1-month (BDI: F=8.25, p<0.01, Epworth (F=11.05, p<0.01).

Conclusions:

Pre-treatment, OSA patients evidenced impaired alertness and memory in comparison to healthy controls. Significant and equivalent improvements in objective measures of alertness/memory and self-reported sleepiness and depression were observed following one-month of treatment with MAD and CPAP therapy; however, OSA patients in both treatment groups remained impaired relative to healthy controls. The AMP is an easy-to-administer method for quantifying OSA-related cognitive impairments and verifying treatment efficacy.

Experiment 5: Autonomic Nervous System Response to Treatment in Patients with Obstructive Sleep Apnea

Introduction:

Increased activity of the sympathetic nervous system (“SNS”) as measured by catecholamine blood levels, muscle sympathetic nerve activity or heart rate variability (“HRV”) has been reported in patients with obstructive sleep apnea (“OSA”). Continuous positive airway pressure (“CPAP”) therapy reduces the activity of the SNS. Mandibular advancing devices (“MAD”) have proven efficient in reducing the number of abnormal respiratory events in mild and moderate OSA. However, little is known regarding MADs efficacy in restoring the balance in the autonomic nervous system. This study investigated the effects of MAD treatment upon the activity of SNS and parasympathetic nervous system (“PNS”) using HRV.

Method:

24 patients and 10 healthy controls with mild to moderate OSA underwent a 3-hour baseline AMP session which included the acquisition of EEG and EKG during a range of visual tasks (e.g., visual discrimination, image recognition) and vigilance tasks. Four quantified neurocognitive factor scores (e.g., sustained attention, processing speed, verbal memory and visuospatial memory); alertness, attention, verbal/visuospatial learning, and memory and sympathetic activation were quantified for differentiation of experts and novices. The patient group underwent a second AMP session after one month of therapy with a mandibular repositioning device.

Spectral analysis of HRV was performed prior to (Session 1) and after one month of MAD treatment (Session 2) in 24 subjects with mild to moderate OSA (pre-treatment AHI=18±11) and 10 healthy individuals (session 1 only) from 5-minute segments of EKG. Differences in Low Frequency (“LF”), High Frequency (“HF”) and LF/HF ratio between OSA patients and healthy controls, and before and after treatment in OSA subjects were tested with t-test for independent and paired samples respectively.

Results:

OSA patients had higher pre-treatment LF and LF/HF ratio compared to healthy controls (LF: t=3.27, p<0.01; HF/LF: t=3.08, p<0.01). A significant decrease in LF/HF ratio was found between the two sessions (t=2.71, p<0.01), accompanied with a decrease in LF (t=2.24, p<0.05) and AHI (10.5±6.9, t=2.51, p<0.01). Post-treatment LF/HF ratio was still higher in OSA patients compared to healthy controls (t=2.18, p<0.05). HF did not change significantly.

Conclusions:

Analysis of EKG provides unique but complimentary information that can be combined with other measures (e.g., EEG, FMRI, MEG, MRI) to provide an improved measure of cognitive function and/or assessing the significance of a cognitive function score. Though preliminary, these findings suggest that the MAD successfully ameliorates the increased activity of the SNS in OSA patients, and provide additional support for MAD therapy as important alternative to CPAP.

### Experiment 6: Psychophysiological Metrics of Experts in Precision Tasks

Introduction:

The execution of tasks that require precision (e.g., firearms marksmanship, golf, etc.) involve the instantiation of a well-defined set of sensory, motor and cognitive skills that can be improved via a combination of classroom instruction and field practice training. Introduction of integrated neuroscience-based evaluation technologies coupled with targeted pre-training intervention accelerates precision related skill acquisition and provide quantitative markers of successful outcomes.

Methods:

A total of 13 USMC-qualified expert marksmen and 32 novices participated in the study using a replica of the M16/A2 rifle which recorded the movement of the muzzle, trigger pressure and break, and shot accuracy. The marksmanship test protocol for both the experts and novices consisted of eight trials of five shots each, shot at a simulated 200 m distance and in the kneeling position. The pacing of shots was not regulated.

Prior to the marksmanship test protocol, subjects underwent a single 3-hour baseline AMP session to delineate neurocognitive and cardiovascular predictors of marksmanship aptitude. The AMP session included the acquisition of EEG and EKG during a range of visual tasks (e.g., visual discrimination, image recognition) and vigilance tasks. Four quantified neurocognitive factor scores (e.g., sustained attention, processing speed, verbal memory and visuospatial memory); alertness, attention, verbal/visuospatial learning, and memory and sympathetic activation were quantified for differentiation of experts and novices.

Results:

Based on the measure of shot group precision, experts exhibited substantially greater levels of visuospatial processing speed and above average ability to sustain attention as compared to a normative database. Experts showed significantly less sympathetic activation during the non-stressful paced button-press Eyes Closed (“EC”) and Eyes Open (“EO”) AMP tests (as measured by the mean low frequency HRV PSD)

These results confirm that experts involved in precision tasks are capable of modulating their physiology to appropriately match task demands. The identification of predictive metrics of expertise during precise tasks opens the door for accelerating novice instruction by using psychophysiological tests to identify novices that may experience difficulty during training.

Conclusions:

Results obtained from a sub-set of tests derived from a 3-hour AMP test battery were able to identify individuals with neurocognitive and cardiovascular capabilities which translate to expertise when performing precise tasks.

In summary, the present method and system operates as follows—a subject is tested at one or more sessions, while performing a cognitive task battery with one or more tasks, during which time physiological signals are recorded. During each test, a subject's behavioral responses and physiological signals are measured as he or she performs a series of repetitions of easy and more difficult sub-tasks designed to assess vigilance, verbal and visuospatial memory, selective and focused attention, distractibility, and learning aptitude, as well as obtaining information during resting conditions. In one embodiment, the present system combines a) a computing device with a screen to present the test information and b) means to obtain the users response with the physiological data acquisition (e.g., brain waves, cardiac activity (EKG), respiration and oxygen saturation, etc.). The test acquisition system may be accessed locally (e.g., using a desk top computer system), under mobile conditions (e.g., using a portable computer, personal digital assistance device, etc.) or in combination with distribution across a network or the internet. A biological sample can be optionally collected while the test is performed so that bio-markers can be subsequently extracted and combined with the testing data.

The present method and system allows analytically derived measures of neurocognitive (e.g., behavioral and physiological), anthropometric and physiological variables or states to be used in assessing a patient's mental state (e.g., drowsiness, anxiety, depression, etc.) or change in mental state (e.g., as a result of pharmacologic intervention, etc.). Additionally, these measures can be referenced to a) normal and abnormal populations, and/or b) in a repeated measures manner. The combination of outputs from the neurocognitive, anthropometric and physiological classifications are further able to be combined into a profile that can based on the results from one or more test sessions.

Benefits associated with the present method and system include, by measuring the brain's electrical activity in combination with cardiac activity and other physiological signals during neurocognitive testing, a comprehensive psychophysiological profile can be compiled efficiently, objectively, with comparative ease on repeated occasions, while accounting for emotional, endocrine, immune or other factors that may otherwise unpredictably impact neurocognitive functions. This differs from current psychometric tests or cognitive task batteries that either do not directly measure brain function, or when they do, do not account for these additional factors that are known to impact cognitive functions. The Interactive Psychophysiological Profile system also differs from prior attempts to use measures of brain activity to characterize cognitive function in that it combines multiple measurements of brain and/or cardiac activity while the subject is both resting and performing a series of tasks designed to assess fundamental cognitive constructs. This combination allows measurement of stress, anxiety and emotional lability measures of task performance, and when information is obtained from analyzed biological samples, measures of immune and endocrine function. In one embodiment, the present system has higher sensitivity (and potentially specificity) to individual differences in fundamental neurocognitive functions or changes in an individual's fundamental neurocognitive functions due to a disease, its treatment, or other causes. With respect to assessing treatment outcome, the system may provide evidence of the benefits of treatment to mental, cardiac and immune function, the comprehensiveness of which impacts recommended treatment options designed to address the identified deficiencies.

Those of skill in the art will appreciate that the various illustrative functions, modules and method steps described in connection with the above described figures and the embodiments disclosed herein can often be implemented as electronic hardware, software, firmware or combinations of the foregoing. To clearly illustrate this interchangeability of hardware and software, various illustrative modules and method steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module or step is for ease of description. Specific functions can be moved from one module or step to another without departing from the invention.

Moreover, the various illustrative modules and method steps described in connection with the embodiments disclosed herein can be implemented or performed with a general purpose processor, a digital signal processor (“DSP”), an application specific integrated circuit (“ASIC”), field programmable gate array (“FPGA”) or other programmable logic device, discrete gate or transistor logic, discrete hardware components, or any combination thereof designed to perform the functions described herein. A general-purpose processor can be a microprocessor, but in the alternative, the processor can be any processor, controller, or microcontroller, similar hardware. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer readable storage media including a network storage medium. An exemplary storage medium can be coupled to the processor such the processor can read information from, and write information to, the storage medium. In the alternative, the storage medium can be integral to the processor. The processor and the storage medium can also reside in an ASIC.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent exemplary embodiments of the invention and are therefore representative of the subject matter which is broadly contemplated by the present invention. It is further understood that the scope of the present invention fully encompasses other embodiments.

What is claimed is:

1. A method of determining a subject's cognitive and emotional state comprising:
obtaining a first physiological measure from the subject which provides baseline data;
administering to the subject a test including a plurality of self-paced computerized tasks while obtaining a second physiological measure from the subject which provides task-related data;
deriving one or more scores quantifying the subject's cognitive and emotional states utilizing the task-related data; and
creating a profile by determining the significance of the one or more scores with reference to normative data from a plurality of subjects.

2. The method of claim 1, wherein the obtaining the first and second physiological measures includes measuring biologically active molecules from the subject.

3. The method of claim 1, further comprising:
deriving or more scores quantifying the subject's immune status utilizing the task-related data.

4. The method of claim 1, further comprising:
providing individualized suggestions for treatment options based on the profile results.

5. The method of claim 1, wherein the first and second physiological measures include electroencephalogram (“EEG”) and electrocardiogram (“EKG”).

6. The method of claim 5, wherein the first and second physiological measures further include at least one physiological measure selected from the group including oxygen levels in the blood, brain, or peripheral tissues; respiration; muscle electrical activity; eye movement; galvanic skin reaction; body temperature; and skin temperature.

7. The method of claim 2, wherein the biologically active molecules are selected from the group including blood, saliva, sweat, urine, and hair.

8. The method of claim 1, wherein the test including a plurality of self-paced computerized tasks assesses a behavior selected from the group including learning speed and aptitude; verbal and visuospatial memory; sustained attention; selective attention; distractibility; impulsiveness; adaptability; flexibility in responding; speed in responding; and propensity for risk-taking.

9. The method of claim 5, wherein the subject's level of alertness, mental effort and engagement during each of the self-paced computerized tasks are characterized by:
measuring on a second-by-second basis the power of the EEG at frequencies from 1 to 40 Hz in 1 Hz steps, and
using a four-class discriminant function to convert these power measures into probabilities that the subject's level of mental effort and engagement belongs to one of the four classes defined as High Engagement, Low Engagement, Distraction and Drowsiness.

10. The method of claim 5, wherein the subject's mental workload is characterized by measuring on a second-by-second basis EEG recorded at frequencies from 1 to 40 Hz in 1 Hz steps using a two-class discriminant function to convert these power measures into probabilities that the subject is at Low or High workload.

11. The method of claim 8, wherein the subject's working memory is characterized by analyzing a distribution of the subject's workload measures during verbal and visuospatial memory tasks.

12. The method of claim 5, wherein the subject's level of stress, anxiety, emotional lability and emotional control are characterized by:
determining the subject's heart rate from the EKG,
computing the spectral indices of heart rate variability in the low frequency range and high frequency range, and
computing the ratio of the low frequency power to high frequency power during each of the plurality of tasks.

13. The method of claim 1, wherein the one or more scores include susceptibility to sleep deprivation, engagement, mental workload, drowsiness level, neurocognitive factor scores, apnea composite evaluation scores and emotional state index.

14. The method of claim 1, wherein the normative data from a plurality of subjects include the scores of the plurality of subjects organized in a database.

15. A method of creating a physiological profile for a subject comprising:
obtaining a first physiological measure from the subject which provides baseline data;
administering to the subject a test including a plurality of self-paced computerized tasks while obtaining a second physiological measure from the subject which provides task-related data;
deriving a plurality of scores quantifying the subject's physiological state utilizing the task-related data;
determining a diagnosis based on the plurality of scores and normative data from a plurality of subjects; and
generating the physiological profile for the subject, the physiological profile including the diagnosis.

16. The method of claim 15, wherein the physiological profile further includes:
suggestions of possible causes of the observed patterns of the scores describing neurocognitive functions and emotional states; and
recommendations about further evaluation procedures or available treatment options for the diseases or states that are presumed causes of the observed patterns of the said descriptors of neurocognitive and/or emotional states.

17. The method of claim 15, wherein the diagnosis includes comparing the subject's plurality of scores to the normative data from a plurality of subjects with and without a disease, disorder or trait.

18. A system for use in determining a subject's cognitive and emotional state comprising:
a sensor in communication with the subject;
a display in communication with the subject; and
a processor in communication with the sensor and display, the processor configured to:
obtain a first physiological measure from the sensor which provides baseline data;
provide to the display a test including a plurality of self-paced computerized tasks while obtaining a second physiological measure from the sensor which provides task-related data;
derive one or more scores quantifying the subject's cognitive and emotional states utilizing the task-related data; and
create a profile by determining the significance of the one or more scores with reference to normative data from a plurality of subjects.

19. The method of claim 18, wherein the system comprises a mobile communication device.

20. The method of claim 18 wherein the subject accesses and completes the test including a plurality of self-paced computerized tasks via the internet.
